(19) United States
(12) Patent Application Publication
(10) Pub. No.: US 2024/0115590 A1
(43) Pub. Date: Apr. 11, 2024

(54) COMPOSITIONS AND THERAPEUTIC METHODS FOR TREATING CHRONIC SEQUALAE FOLLOWING VIRAL INFECTIONS

(71) Applicant: Jean-Jacques Schoch, Van Nuys, CA (US)

(72) Inventor: Jean-Jacques Schoch, Van Nuys, CA (US)

(21) Appl. No.: 18/203,640

(22) Filed: May 30, 2023

Related U.S. Application Data
(60) Provisional application No. 63/348,002, filed on Jun. 1, 2022.

(51) Int. Cl. A61K 31/706; A61K 9/00; A61K 31/09; A61K 31/12; A61K 31/198; A61K 31/352; A61K 31/409; A61K 31/675; A61K 33/08; A61K 33/30; A61K 36/068; A61K 36/07; A61K 36/074; A61K 36/185; A61K 36/19; A61K 36/23; A61K 36/258; A61K 36/28; A61K 36/48; A61K 36/481; A61K 36/52; A61K 36/71; A61K 36/81

(52) U.S. Cl. CPC A61K 31/706; A61K 9/0095; A61K 31/09; A61K 31/12; A61K 31/198; A61K 31/352; A61K 31/409; A61K 31/675; A61K 33/08; A61K 33/30; A61K 36/068; A61K 36/07; A61K 36/074; A61K 36/185; A61K 36/19; A61K 36/23; A61K 36/258; A61K 36/28; A61K 36/48; A61K 36/481; A61K 36/52; A61K 36/71; A61K 36/81

(57) ABSTRACT

Embodiments of therapeutic protocols to treat Post-Acute Sequelae SARS-CoV-2 infection (“PASC”), a.k.a. “long Covid,” are described. The PASC treatment protocols focus on a moderating a hyperimmune response; destroying and removing the SARS CoV-2 spike protein from the gut and body, detoxifying the body and the brain; replenishing key nutrients; mitigating depression and anxiety; and a regimen of physical and mental exercises. A standard six-week protocol and a shorter, three-week protocol are disclosed for those with a milder form of PASC are disclosed.

### BACKGROUND OF THE INVENTION

### Technical Field

The disclosures herein relate to therapeutic methods of treating chronic sequelae of viral infections. Specifically, the disclosed invention relates to compositions and treatment protocols for treating chronic illness following infection with the novel coronavirus SARS-CoV-2 (“Covid.”)

### State of the Art

“Long Covid” or “Post-Covid Syndrome” is a set of symptoms that vary somewhat between individuals. People who have it are called “Long Haulers.” Its clinical name is Post-Acute Sequelae SARS-CoV-2 infection, or PASC. As much as fifty percent of people infected with the SARS-CoV-2 virus (Covid-19) develop PASC. The exact cause of PASC is unknown, however it behaves like an autoimmune-like disease and can be fully disabling. Currently, there are no known cures or effective treatment protocols to facilitate recovery from PASC with full symptom resolution. There is also no known useful understanding of PASC symptoms.

Because of these and other problems, there is a need for compositions and treatment protocols for treatment of PASC that address the aforementioned deficiencies.

### BRIEF SUMMARY OF THE INVENTION

The disclosures herein relate to a breakdown of PASC symptom complexes into specific groups based upon causative factors. An outline of novel therapeutic protocols for addressing each symptom complex group is then provided. Compositions useful for treating each symptom complex are discussed. Dosing regimens for a full six (6) week therapeutic protocol are disclosed in detail in the written description and drawing figures and a rationale for each is provided. An abbreviated protocol for treating mild PASC is also disclosed.

The foregoing and other features and advantages of the invention will be apparent to those of ordinary skill in the art from the following more particular description of the invention and the accompanying drawings.

### BRIEF DESCRIPTION OF THE DRAWINGS

FIG. 1 is an illustration diagramming symptom complex groups of PASC (Long Covid);

FIG. 2 is a chart correlating causative factors and therapeutic compositions with symptom complex groups of PASC;

FIG. 3 is an illustration diagramming therapeutic compositions for treating each of the symptom complex groups of PASC;

FIG. 4 is an illustration showing therapeutic compositions for eradicating SARS CoV-2, spike proteins, and other virus remnants from the body and gut microbiome;

FIG. 5 is a chart showing details of a six (6) week therapeutic protocol for treating PASC;

FIG. 6 is an illustration showing additional details of a six (6) week protocol for treating PASC; and

FIG. 7 is an illustration showing an abbreviated three (3) week protocol for treating mild cases of PASC.

### DETAILED DESCRIPTION

Various example embodiments of compositions and treatment protocols for treatment of PASC are disclosed herein. The therapeutic protocols comprise specific regimens to reverse damage caused by PASC and resolves symptoms targeting at least six (6) specific mechanisms of damage occurring in human patients with PASC.

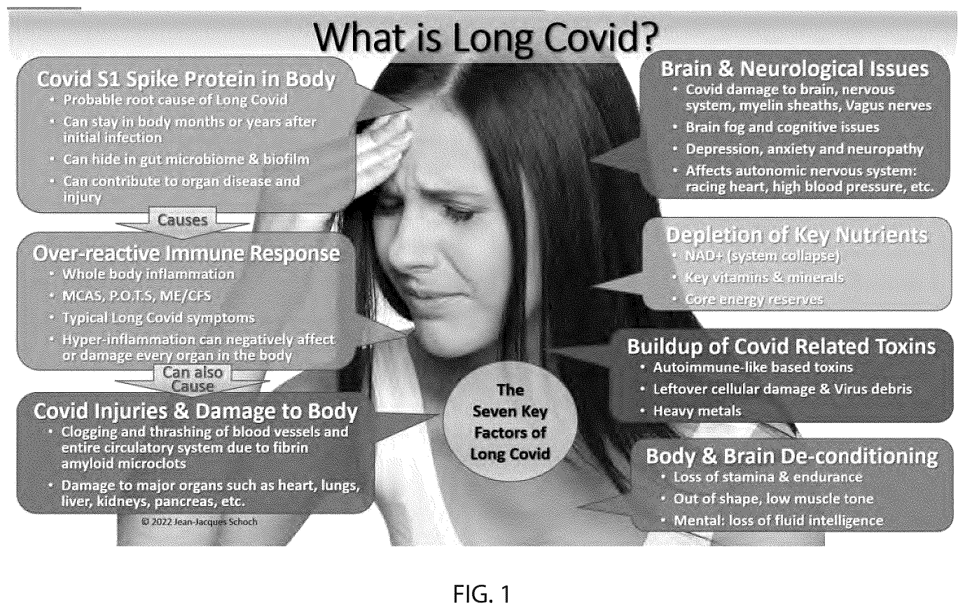

FIG. 1 is an illustration diagramming symptom complex groups of PASC (Long Covid). The root cause of PASC is thought to be triggered by a ribonucleic acid (“RNA”) remnant of the Covid-19 virus that remains in the body-even 15 months or longer after the initial Covid-19 infection has resolved. According to recent research, traces of the Covid-19 S1 spike protein remains in the body, brain, and gut microbiome post-infection. It is probable that the S1 spike protein triggers the body's immune system to overreact when over-exerting or over-stressed. This releases a storm of cytokines, chemokines, histamines, fractalkines, etc., causing full-body hyper-inflammation and many PASC symptoms.

Online videos, websites, and even a few doctors have recommended the antiparasitic pharmaceutical drugs Ivermectin and Hydroxychloroquine to address PASC. But after more than 18 months of testing and research, there are no peer-reviewed studies that show these drugs by themselves to be any more effective than a placebo in healing PASC.

For Covid related issues, Ivermectin's only value appears to be as a zinc ionophore; in that it helps push zinc into cells to prevent those cells from becoming infected. However, an over-the-counter supplement, Quercetin, is also an effective zinc ionophore, but is completely safe and doesn't ruin the gut microbiome, like Ivermectin does.

It is now believed that a majority of people who were infected with Covid-19 have lingering effects, from loss of taste and smell to severe disability. It is possible that over 50% of people who have had Covid-19 are either completely disabled or unable to fully function in life (work, school). That could mean that up to 25 million Americans and 140 million people worldwide are affected long-term by PASC.

Researchers have also identified three other risk factors that can lead to PASC: (1) the presence of specific autoantibodies that attack the body, (2) Epstein-Barr virus DNA levels in the blood, and (3) pre-existing Type 2 diabetes. As of now, a medical consensus on how to heal people with PASC is lacking. In other words, there is nothing conventional medicine can offer, medically or pharmaceutically, to completely remedy PASC. All a healthcare provider can currently recommend is a low-histamine diet, some antihistamine drugs and steroids, plenty of rest and sleep, and avoiding overexertion and stress.

Therapeutic protocols for healing PASC are set forth in two parts. First, these disclosures organize PASC into seven key symptom complexes and addresses treatment of each symptom complex separately. Breaking down PASC into separate symptom complexes is a key feature of realistic opportunities to heal PASC. Second, after defining the seven key symptom complexes, therapeutic protocols are set forth based upon a holistic, whole-body approach that allows for the discovery of requirements to fully heal PASC. Six specific therapeutic regimens are provided that synergistically address each of these seven key factors, thereby allowing for complete, full-body healing.

### PASC Symptoms Result from Seven Key Factors

As shown in FIG. 1, the symptoms of PASC are organized by seven key factors:
(1) Covid S1 Spike Protein remains in the body for weeks, months, or even years beyond the initial infection. This can adversely affect health and cause injury to major bodily organs. The spike protein and possibly other virus remnants remaining in the body are the root cause of PASC.
(2) Over-reactive immune response to the Covid S1 spike protein and possibly other virus remnants causing whole body inflammation, hair loss, brain fog, depression, anxiety, mast cell activation syndrome (MCAS), postural orthostatic tachycardia syndrome (POTS), Myalgic Encephalomyelitis/Chronic Fatigue Syndrome (MF/CFS) and can negatively affect or damage every organ in the body.
(3) Covid-related injuries & damage to major organs, typically manifesting as diseases that are circulatory, neurological, heart, lungs, digestive, diabetic, etc.
(4) Brain and neurological issues, causing depression, anxiety, brain fog, neuropathy, and lost memories. These can also negatively affect the autonomic nervous system contributing to postural orthostatic tachycardia syndrome (POTS).
(5) Depletion of key nutrients, and collapse of the NAD+ (Nicotinamide adenine dinucleotide) system, causing general malaise, exhaustion, slowness to heal, depression, and exacerbating PASC symptoms
(6) Buildup of Covid-related toxins in the body, for example, virus remnant residue, autoimmune-like toxins, leftover cellular damage debris, and heavy metals. These can also cause malaise.
(7) Body and brain de-conditioning, resulting in loss of stamina & endurance, being out of shape with low muscle tone, and mental: loss of fluid intelligence.

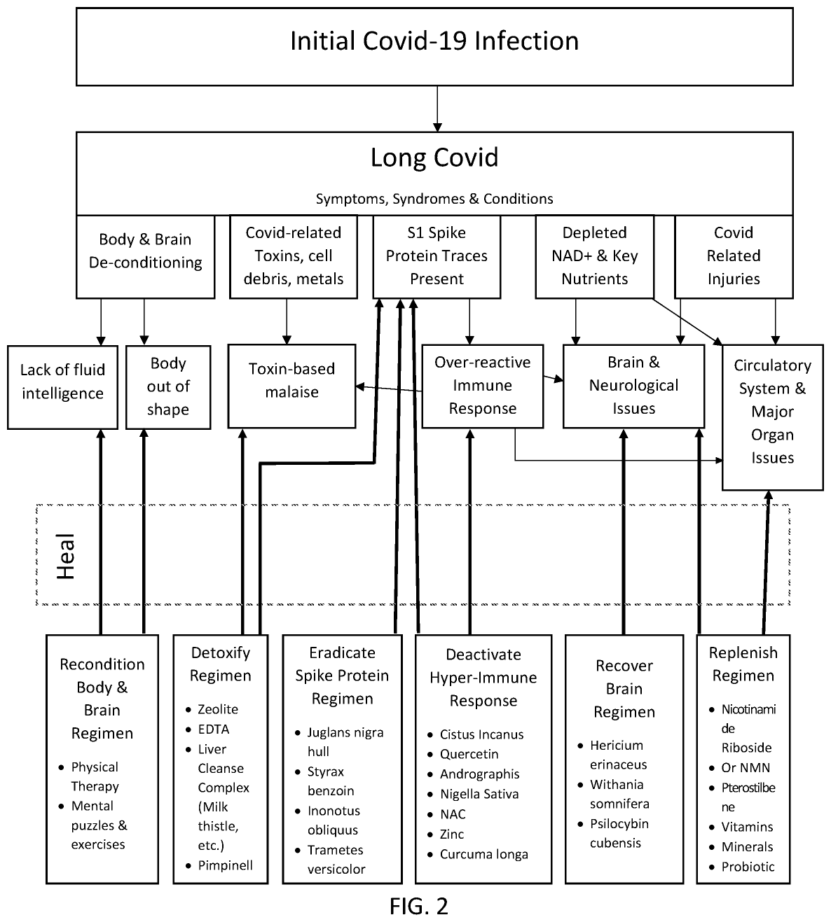

FIG. 2 is a chart correlating causative factors and therapeutic compositions with symptom complex groups of PASC. As shown in FIG. 2, seven of these factors are addressed with a combination of therapeutic protocols, leading to a successful full-body recovery from PASC. These protocols together forma a comprehensive and holistic collection of highly researched and tested therapies designed to help heal all seven fundamental factors of PASC. Here is an overview of these protocols:

### Protocol 1—Eradicate Spike Protein

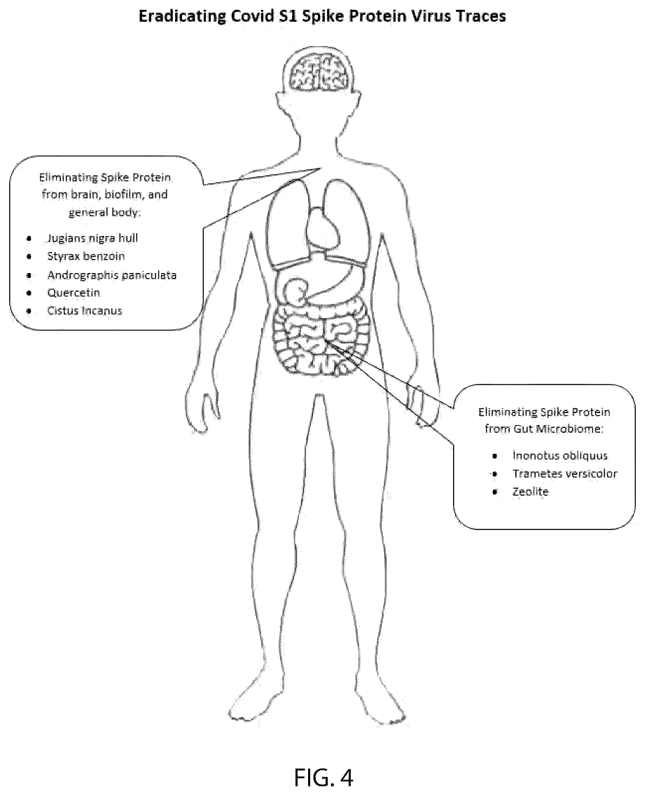

FIG. 4 is an illustration showing therapeutic compositions for eradicating SARS CoV-2, spike proteins, and other virus remnants from the body. This can include the brain, ovaries, and other sex organs, sinus cavities, gut microbiome, lymphatic system, and other yet unknown body areas. Ridding the gut and body of the SARS CoV-2 S1 spike protein traces and other possible virus remnants that remain in the body post initial covid infection. This is addressed using the following regimen:
1. Juglans nigra hull
   i. Works synergistically with Styrax benzoin to eradicate the spike protein remnants from the body, by likely “melting” the fatty virus capsid.
2. Styrax benzoin
   i. Works synergistically with Juglans nigra hull to eradicate the spike protein remnants from the body, by likely “melting” the fatty virus capsid.
   ii. Works to dissolve the biofilms where the spike protein can hide and be protected.
3. Inonotus obliquus
   i. Works synergistically with Trametes versicolor to adjust the Gut Microbiome in such a way that it causes the spike protein remnants to no longer be able to hide amongst the gut microbiome.
4. Trametes versicolor
   i. Works synergistically with Inonotus obliquus to adjust the Gut Microbiome in such a way that it causes the spike protein remnants to no longer be able to hide amongst the gut microbiome.
5. Quercetin
   i. Aids in the elimination of the Covid S1 Spike protein by significantly reducing inflammation and oxidants.
6. Andrographis paniculata
   i. Has broad-spectrum antiviral properties that both reduce the viral activity and over-reactive immune response, as well as aiding in the eradication of the spike protein remnants.
7. Cistus incanus
   i. Has broad-spectrum antiviral properties that aids in the elimination of the SARS CoV-2 S1 spike protein

### Protocol 2—Deactivate Hyperimmune Response

Reducing and/or eliminating the over-reactive immune response to the SARS CoV-2 S1 spike protein and possibly other virus remnants. This is addressed using the following regimen:
1. Nigella sativa
   i. Has strong antiviral properties.
   ii. Helps reduce the hyper-inflammation throughout the body.
   iii. Helps eliminate the histamine and cytokine storms ravaging the body (Mast Cell Activation Syndrome) caused by the over-reactive immune response.
2. Curcuma longa
   i. Helps reduce the hyper-inflammation throughout the body.
3. N-Acetyl-L-Cysteine (NAC)
   i. Precursor to Glutathione, a powerful antioxidant which helps eliminate the barrage of oxidative debris flooding the body from the autoimmune-like reaction to the spike protein.
4. Quercetin
   i. Helps eliminate the histamine and cytokine storms ravaging the body (Mast Cell Activation Syndrome) caused by the over-reactive immune response.
   ii. Significantly reduces the over-reactive immune response
   iii. A powerful anti-inflammatory that reduces severe inflammation, as well as an antioxidant and analgesic (pain killer)
   iv. Acts as a Zinc ionophore, which helps zinc transfer more easily into cells protecting them from further damage and infection.
5. Zinc
   i. Used to protect cells from damage and infection
6. Andrographis paniculata
   i. Has broad-spectrum antiviral properties that both reduce the viral activity and over-reactive immune response, as well as aiding in the elimination of the spike protein remnants.
7. Cistus incanus

### Protocol 3—Replenish & Heal

Replenishing bodily nutrients and to help heal Covid-related injuries & damage to body and major organs.
1. Nicotinamide Riboside
   i. Helps rebuild the NAD+ (Nicotinamide adenine dinucleotide) system which is critical for cell protection and cell rejuvenation.
   ii. Nicotinamide mononucleotide (“NMN”) is a viable alternative to nicotinamide riboside.
2. Pterostilbene
   iii. Powerful antioxidant that also increases the effect of Nicotinamide Riboside or NMN in rebuilding the NAD+ system
3. Panax quinquefolius
   i. Increases circulation and vascular function
   ii. Has antioxidation, anti-inflammatory, vasorelaxation, antiallergic, antidiabetic, and anticancer properties.
   iii. An adaptagenic that helps raise the body's depleted core energy levels
4. Probiotics
   i. Helps rebuild the gut microbiome, which is critical for healing and the ultimate removal of the spike protein.
5. Key Vitamins and minerals known to either be depleted by Covid-19, or that aid in the recovery of PASC:
   i. Vitamin C
   ii. Vitamin D
   iii. Vitamin B Complex
   iv. Pantothenic acid (B5)
   v. Chlorophyll
   vi. Magnesium
   vii. Potassium
   viii. Selenium

### Protocol 4—Recover Brain

Healing brain and neurological issues while reducing brain fog, depression, and anxiety with the following regimen:
1. Hericium erinaceus
   i. Has neurogenesis capabilities
   ii. Induces nerve growth factor with Erinacines (cyathin diterpenoids), its bioactive ingredient.
   iii. Creates protection against Ischemic Stroke, Parkinson's Disease, Alzheimer's Disease, dementia
   iv. Reduces depression, neuropathy and neuropathic pain,
ii. Withania somnifera
   i. It has a Cognition Promoting Effect, and reverses memory loss
   ii. Can reverse neurodegenerative diseases such as Parkinson's, Huntington's and Alzheimer's diseases.
   iii. Has a GABA (Gamma-Aminobutyric Acid) like effect and can promote formation of dendrites. (Neurogenesis)
   iv. Reduces stress, increases stamina,
   v. An adaptagenic that helps raise the body's depleted core energy levels
iii. Psilocybin cubensis
   i. Uses neurogenesis capabilities to help rebuild damaged brain neurons, and overall neurological damage
   ii. Uses neuroplasticity capabilities to replace old fear “circuits” to significantly reduce depression, anxiety, and PTSD.

### Protocol 5—Detoxify

Detoxifying the body and brain from Covid-related toxins, and toxins released via viral cleanup:
1. Zeolite clinoptilolite
   i. Performs reversible ion exchange and has a powerful adsorption capacity. Attracts and captures positive-charged debris and remnants, essentially removing metals, virus remnants, cell debris, and leftover oxidants left over from the initial Covid infection, from the intestinal tract and body.
2. Disodium ethylene diamine tetra-acetic acid
   i. Used for a final chelation of heavy metals
3. Pimpinella anisum
   i. Used to help detox the brain
4. Liver Cleanse complex:
   i. Desmodium molliculum
   ii. Guggulsterones Commiphora mukul
   iii. Phosphatidyl Choline
   iv. Sllymarin, Slybum marianum
   v. Dandelion Root
   vi. L-Methionine
   vii. Cynara scolymus
   viii. Arctium lappa
   ix. Hydrangea arboresescens
   x. Achillea millefolium

### Clinical Example

The inventor/applicant, a 63-year-old healthy, active adult male, contracted Covid, which was confirmed by a Covid-19 RT-PCR test on Nov. 29, 2020. Before becoming ill, he walked three miles a day, went to the gym, and bicycled several times a week. He ate a healthy, Mediterranean-based diet. Although he would get the occasional colds with runny nose and sore throat, he hadn't experienced the flu in about 30-plus years. He characterized himself as “a healthy person with a naturally strong immune system.”

A few days before Thanksgiving in 2020, before any vaccines were available, his 27-year-old son, who was staying with him during the pandemic, said he was not feeling well and started coughing a bit. Within 2 days, the patent's partner and he were beginning to feel the symptoms of Covid. The all tested positive for the Covid-19 infection. The next nine days he experiences moderately severe symptoms, including chest pain and pressure, massive headaches, random shooting pains body pains, and severe malaise. His partner experienced similar symptoms. The patient further characterized his experience:
Having Covid was difficult. I had never been so physically stressed. At times, it felt like a 300 lb. person was sitting on my chest. My heart was racing. It was difficult to sleep at night with my heart pounding so loud I could hear it in my head. The bad headaches were nonstop and no over-the-counter pain medications would relieve them. I never had a high fever, just a slight temperature of 99.1 and a very light cough. We took it day-by-day. I didn't know if it was going to get worse—or if I was going to have a heart attack.

While ill, he self-medicated with supplements and drugs that were at that time being recommended while going through Covid. These included: Pepcid AC, Zyrtec, melatonin, aspirin, vitamin C, vitamin D, zinc, and quercetin. On the tenth day, he started feeling better and slowly resumed his daily walks over the next week. At first it was a mile and then he increased it to two miles. The only immediate lingering effects he experienced were some memory loss (he couldn't remember his multiplication tables and certain pleasant childhood memories), “feeling down,” general body aches, and symptoms of peripheral neuropathy. He is a musician and after Covid he could barely play piano due to what he interpreted as nerve damage in his hands and fingers. He remained optimistic, despite these lingering symptoms. He felt he had not only survived Covid, but it felt like I was going to “get back to [his] normal self.”

Roughly two weeks later, however, his symptoms returned. Starting in mid-December, 2020 he again began to feel chest pains while walking outside and a constant headache that would not go away with ibuprofen, acetaminophen, naproxen, or aspirin.

Because of these symptoms, his doctor recommended that he rest, not do any strenuous physical activity, avoid emotional stress—basically do nothing. This worked for a few weeks and he was slowly starting to feel slightly better.

Although he did not feel strong enough to return to work or resume daily exercise, he felt he could organize and clean his home, and similar light chores. These activities seemed to make his symptoms much worse. In some ways, he felt worse than during the active infection. This gradually worsened over the next month. Over the next five months, he reported the following intermittent symptoms (in his words):
Massive migraine-like headaches that nothing would relieve
“Lung Burn” a term PASC sufferers call the weird, intense chest pains
Racing heart—my resting heart rate was 115 BPM (versus normal 65 BPM)
High blood pressure, averaging 141 over 111 (versus normal 117 over 73)
Brain fog—with severely missing memories
Inability to speak—it was extremely difficult to formulate a thought and even harder to try speaking a thought; I often stuttered when I spoke
Extreme depression and anxiety
Random shooting pains throughout my body, similar to a bee sting
Hair loss—clumps of hair would fall out when I showered

He felt as though “Covid had turned me from a vibrant, bright, athletic person into a feeble old man, and that alone was an extremely depressing thought.” Nine months into suffering PASC, he was feeling so bad, thinking he was going to have a heart attack or stroke that at one point, that his partner drove him to the emergency room at a local hospital. A series of tests, including EKG and a chest x-ray, were all normal, which is often the case with PASC patients. A few weeks later, he followed up the ER visit with an appointment to see his primary doctor. Additional bloodwork was run with normal test results. He continued to feel physically awful, emotionally frustrated, and desperate to find a solution.

Over the next several months, he sought to fully understand his illness and consider possible alternative therapies, learning that:
1. MRIs, X-rays, blood work and lab tests usually show that everything looks normal in Long Covid patients.
2. Many doctors still do not understand Long Covid and often suggest to the patient that they were just imagining the symptoms.
3. Long Covid seems to come in cycles of feeling lousy, then experiencing some good days, and then back to feeling lousy again; over and over again.
4. Deep breathing exercises help reduce headaches and gave me more energy.
5. Acupuncture helped lower my heart rate and blood pressure.
6. Acupressure, Yoga and Qi Dong* have helped many people feel better. *Qi Gong is a system of coordinated body-posture and movement, breathing, and meditation used for the purposes of health, spirituality, and martial-arts training.
7. A low-histamine diet has made a big difference in many people with Long Covid (it had no benefit for me, probably due to the “Deactivate Autoimmune-like Reaction” supplements I was already taking as part of the protocol).
8. A multi-vitamin with certain key supplements called Myovite® was helping a lot of people. I tried it and it worked really well for me.

He considered a link between a chronic inflammatory state and the immune response, he considered alternative therapies for Lyme disease:
About 3 weeks into having Long Covid, I got the wild idea that maybe some of the herbal Lyme Disease protocols could help me, so I tried the ones created by Buhner, Zhang, and Cowden. They all seemed to help me feel a little better, but they didn't stop the ongoing relapses and ended up not healing Long Covid. I did end up using some of the individual supplements from these Lyme protocols, specifically Andrographis, Stevia Rebaudiana, Desmodium Molliculum (Burbur), and Pimpinella Anisum (Pinella). I chose these based on what truly helped me feel better through trial and error on myself.
And wow! After a few days, I could feel a huge difference in my energy levels and a dramatic lessening of my headaches. This tea was truly helping me a lot.
Whenever I would overdo it and feel relapse symptoms creeping back, I would drink extra amounts of the tea. Often, I would drink two to four cups a day depending on how much I felt I needed it.

He did independent research:
I also started reading online medical journals about the effects of Covid and Long Covid on the body. I learned several things, among them:
1. Many PASC patients have similar symptoms to POTS (postural orthostatic tachycardia syndrome). This is where the autonomic nervous system, which controls involuntary functions such as heart rate, blood pressure and vein contractions that assist blood flow, is no longer working correctly. This can cause the body's blood flow to be as low as 50% of normal blood flow, which can cause a racing heart, deep exhaustion, high blood pressure, headaches, brain fog, etc.
2. Covid-19 can ravage and clog up your blood vessels (arteries, capillaries, veins) that cause the heart to have pump much harder due to the added friction of having to push blood through damaged blood vessels that are cut up and thrashed on the inside, and clogged up due to fibrin amyloid microclots. This was one of the reasons for my racing heart. I also learned that the body will naturally heal these damaged blood vessels over time, especially with proper nutrition.
3. Covid-19 is now viewed as a neurological disease, where Covid affects the brain and nerve sheaths of the body. This contributes to the headaches, brain fog, memory loss, etc.
4. Many PASC patients have MCAS (mast cell activation syndrome) symptoms, where Histamine and Cytokine storms are ravaging the body in much the same way an autoimmune disease does. This can cause inflammation of the brain and other organs.
5. Many PASC patients have Vagus Nerve Inflammation, which can affect proper lung function, heart rate, digestion, and much more.
I had Vagus Nerve stimulation therapy for 10 days, but it tended to worsen my headaches, so I stopped it.
6. Having a Covid infection can significantly lower NAD+ levels in the body, especially as you get older. NAD+ (nicotinamide adenine dinucleotide), a coenzyme that is critical in fighting off viruses and having vital energy levels. It's involved in multiple cellular reactions for the generation of energy and maintenance of cellular health, including immune responses to viral infections.
It is now understood that older people have significantly lower NAD+ levels than the younger, which is one of the major reasons seniors had a higher mortality rate when experiencing a Covid-19 infection.
Low NAD+ levels can also significantly slow down the healing process which makes it very difficult for your body and brain to properly heal from all the damage caused by both the initial Covid infection and then PASC.
7. Low NAD+ levels can also lead to chemical-based depression since your body appears to have a more difficult time creating serotonin, a key brain drug that helps keep you happy and stable. After researching NIHs database on NAD+ I learned that there are three types of supplements that can help raise NAD+ levels. From that research, I learned that Nicotinamide Riboside is the most powerful of these supplements; it increases your NAD+ levels faster than anything else, but it's also very expensive. The next most potent one is NMN or Nicotinamide Mononucleotide, which is somewhat less expensive. The slowest but least expensive method of raising your NAD+ levels is with Nicotinic Acid, the niacin that causes flushing (reddening) in the face and body.
I also learned that adding Pterostilbene to the Nicotinamide Riboside significantly increases the concentration of NAD+ levels in the body.
8. Having a Covid infection can deplete key vitamin, chemical and mineral levels in the body, especially Vitamin D, Selenium, and Folate, which slows down the healing process and affects your energy levels.

Following this research, he felt he better understood the etiology of his symptoms:
Now all my symptoms were starting to make sense. Based on this new knowledge, I started to perform research to see if my symptoms were like other known conditions or diseases (such as POTS, MCAS or low NAD+), and to learn what is used to treat these diseases.
I found some research articles on the NIH database about Black Seed Oil and Quercetin (with Zinc) alleviating symptoms of MCAS (mast cell activation syndrome). I ordered both of those online and within a few days of using them, I again noticed an increase in energy and I was having fewer headaches.
I learned that a Chinese herb, Andrographis could also help suppress the effects of viruses and pathogens. After trying both, I discovered they likewise contributed to me feeling even better.
I also learned that N-Acetyl-L-Cysteine (NAC) is the most powerful precursor to Glutathione which is the only antioxidant made in the body, and the only antioxidant that science knows works intracellularly. Based on this science I started taking it.

These combined actions led to a substantial decrease in his chronic symptoms, most notably a return of his normal sense of smell and improving energy levels. He increased duration and frequency of mild to moderate exercise, particularly gardening and walking outdoors.

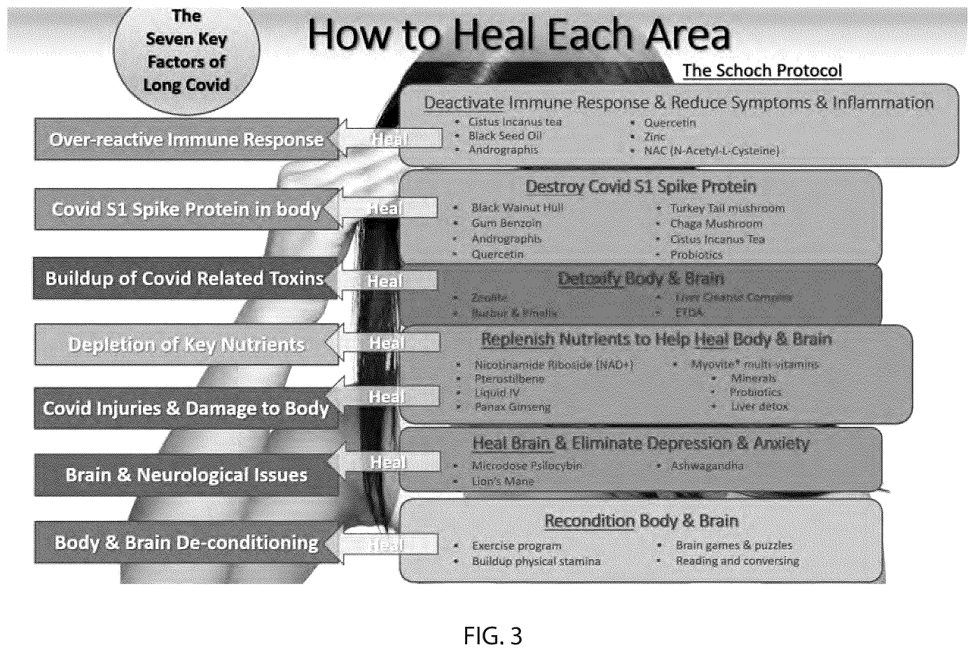

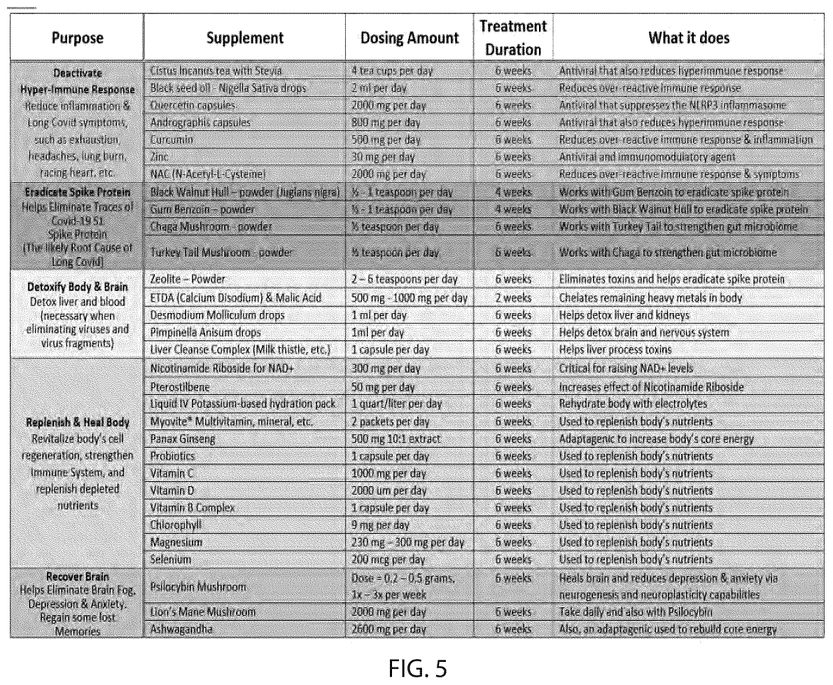

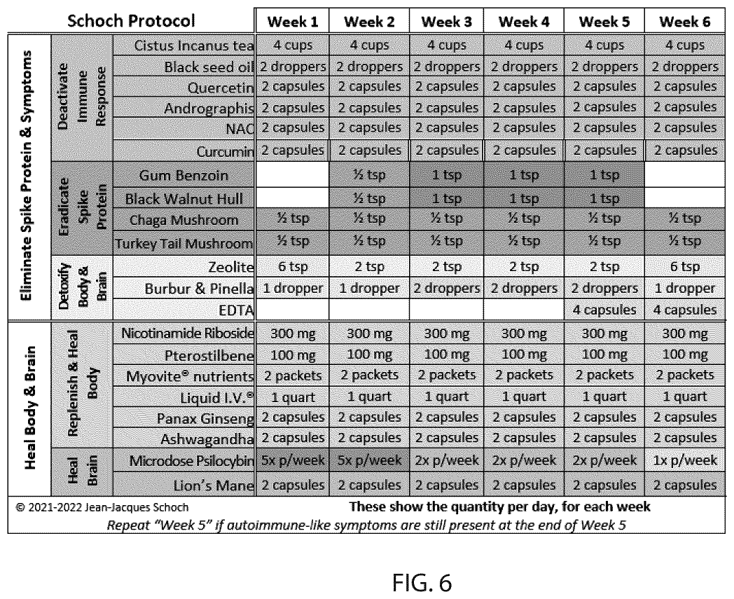

Accordingly, a therapeutic protocol for treating PASC comprises oral consumption of multiple supplements and active substance for a duration of about six (6) weeks. FIG. 3 is an illustration diagramming therapeutic compositions for treating each of the symptom complex groups of PASC. FIG. 5 is a chart showing details of a six (6) week therapeutic protocol for treating PASC. FIG. 6 is an illustration showing additional details of a six (6) week protocol for treating PASC.

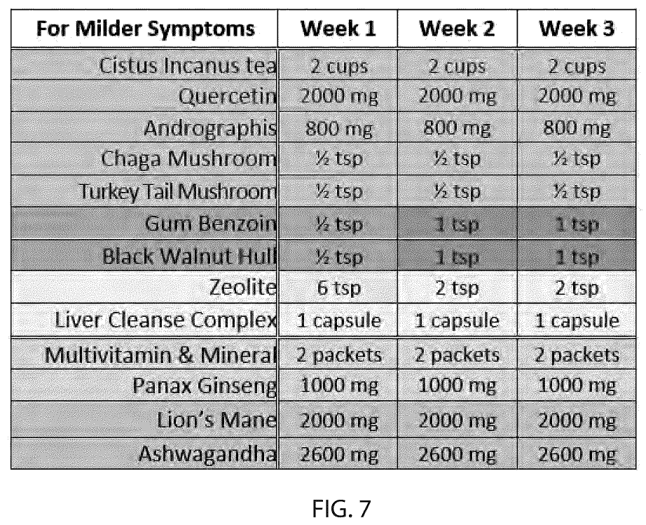

FIG. 7 is an illustration showing an abbreviated three (3) week protocol for treating mild cases of PASC. As shown in FIG. 7, in some embodiments, the therapeutic protocol duration is about three (3) weeks. The shorter, three-week protocol is sufficient for persons with milder symptoms, such as less impact on daily activities, retention of smell and taste, occasional increased fatigability, and frequent (but minor) headaches.

In some embodiments, a six (6)-week therapeutic protocol for treating PASC comprises a mixture of Cistus incanus tea, Black Seed oil, Andrographis, quercetin, zinc (via Myovite®), and N-Acetyl-L-Cysteine (NAC).

In some embodiments, a therapeutic protocol for treating PASC comprises NAC and Ashwagandha. In some embodiments, the NAC is greater than about 1,000 milligrams (mg). In some embodiments, the Ashwaganda is greater than about 1,300 mg. In some embodiments, a therapeutic protocol for treating PASC comprises Nicotinamide Riboside (or NMN), to rebuild NAD+ levels, Pterostilbene, Liquid I.V.® to fully rehydrate and raise electrolyte (particularly, potassium) levels, Panax Ginseng, a probiotic, Vitamin C, and Vitamin D.

In some embodiments, a therapeutic protocol for treating PASC comprises altering the gut microbiome by ingestion of Turkey Tail mushrooms (Trametes versicolor) and Chaga Mushrooms (Inonotus obliquus).

In some embodiments, a therapeutic protocol for treating PASC comprises blunting the chronic immune response by ingestion of Andrographis, Quercetin and Cistus Incanus water-based infusion (“tea”).

In some embodiments, a therapeutic protocol for treating PASC comprises daily oral consumption of a mixture of different mushrooms, including Reishi, Shiitake, and Maritake genera.

In some embodiments, a therapeutic protocol for treating PASC is used in a method to treat chronic symptoms following or during infection with a Herpesvirus. In some embodiments, the Herpesvirus is an Epstein-Barr virus.

In some embodiments, a six-week therapeutic protocol for treating PASC comprises a treatment to moderate a hyperimmune response; a treatment to destroy SARS CoV-2 spike protein, a treatment to detoxify the body and the brain; a treatment to replenish key nutrients; a treatment to mitigate depression and anxiety; and a regimen of physical and mental exercises.

The treatment to moderate the hyperimmune response, in some embodiments, comprises oral administration of a composition of Cistus Incanus tea, Andrographis, Black Seed Oil, Quercetin, n-acetyl cystine (“NAC”), and Zinc. In some embodiments, the treatment additionally includes Quercetin.

The treatment to destroy SARS CoV-2 spike protein, in some embodiments, comprises initial oral administration of Chaga and Turkey Tail mushrooms in a combination with Cistus incanus, Andrographis, and Quercetin. In some embodiments, the treatment additionally comprises oral administration of Gum Benzoin and Black Walnut Hull following a period of treatment with the Chaga and Turkey Tail mushrooms, Cistus incanus, Andrographis, and Quercetin. In some embodiments, the treatment comprises daily administration of Turkey Tail and Chaga mushrooms for seven (7) days, followed by addition of half-doses of Gum Benzoin and Black Walnut Hull for an additional seven (7) days, followed by adding a dose of NAC and increasing the half-doses to full doses for an additional period of twenty-one (21) days.

The treatment to detoxify the body and the brain, in some embodiments, comprises oral administration of Burbur, Pinella, Zeolite, EDTA and a proprietary liver cleanse multivitamin complex (i.e., Myovite®) for about fourteen (14) days. Additional details of this treatment include with the Zeolite, start on a strong triple dose of two teaspoons, three times a day. Take this large amount of Zeolite for just one week so it can do a major body cleanup in the beginning and help mop up whatever heavy metals, loose virus remnants and any other debris remain from the original Covid damage. This larger amount of Zeolite will also work in partnership with the Chaga and Turkey Tail mushrooms to help clean-out the spike protein in the gut. In some embodiments, the treatment comprises reducing the Zeolite to just two teaspoons/day, after two weeks, for a period of four weeks.

Starting about week five, start taking EDTA for the final two weeks to help chelate any heavy metals still in your system as part of the final cleanup. By week six, increase the Zeolite to six teaspoons per day to help mop up any remaining residue from the virus cleanup. In some embodiments, the treatment additional comprises oral administration of Zeolite about once every two weeks.

The treatment to replenish key nutrients, in some embodiments, comprises oral administration of Myovite®, nicotinamide riboside (or NMN) and pterostilbene (for NAD+), Ginseng, Ashwagandha and Liquid I.V.®. During inflammation and symptom reduction, you can start to rebuild and heal the injured areas and restore depleted vitamins, minerals, and other vital nutrients that Covid leaches from the body during the original infection. Additionally, adding collagen peptides, fish oil, and creatine and the proprietary vitamin supplement is helpful to speed up healing. If you're over the age of 50, adding 50 mg of DHEA to boost hormone levels can potentially help the body heal faster. In some embodiments, this treatment is continued for at least a full six (6) weeks. In some embodiments, this treatment can be continued indefinitely to improve a feeling of physical well-being.

The treatment to mitigate depression and anxiety, in some embodiments, comprises Psilocybin micro-doses with Lion's Mane and niacin, and Ashwagandha. Part of the healing process is giving your brain and central nervous system the neurogenesis and neuroplasticity tools it needs to repair the neurological damage caused by Covid. In some embodiments, the treatment comprises an initial course of oral micro-dosing Psilocybin for five consecutive days for the first two weeks. A noticeable improvement in brain function typically results. The Psilocybin dose frequency is then reduced to twice a week starting on week three. On week six, the dose frequency is reduced to once weekly. In some embodiments, the Psilocybin micro-dosing is continued for an additional three (3) days following four (4) days with no Psilocybin micro-dosing. In some embodiments, the additional of a daily meditation practice, for about fifteen (15) to twenty (20) minutes, is added to the Psilocybin micro-dosing.

The regimen of physical and mental exercises, in some embodiments, comprises going on very short walks; 100 feet the first few days, then increasing the distance slowly as tolerated to eventually walking at least one mile daily. Working the arms with light weights is additionally useful. Five-to-fifteen-pound weights are sufficient.

In the final weeks, once the spike protein and over-reactive immune response of PASC are cleared out of the system and resolved, a final cleanup and detoxification combined with deeper reconditioning the body and brain and rebuilding the body's core energy reserves is useful.

Additional dietary supplements are helpful following the completion of all treatments and protocols. These additional supplements include Amazing Grass Greens Blend Superfood™ that is added to a daily shake. This Green powder is nutritious but its best to have alongside live greens (romaine, spinach, spring mix, microgreens, raw celery, etc.). Daily supplements additionally include Fish Oil capsules, DHEA, creatine powder, Vital Proteins Collagen Peptides™, Living Silica Collagen Booster™, and NAC. Additional daily supplements may include Nattokinase and French maritime pine bark extract. A diet low in histamine may be helpful in some individuals.

Some foods help diminish symptoms of PASC, including protein shakes with a half-cup of mixed berries and a half a frozen banana; leafy green salads with tomatoes, scallions, radishes, avocado, olive oil and apple cider vinegar; salmon and other seafood; poultry such as chicken and turkey; fruits such as apples, cherries, raspberries blackberries, and bananas; and vegetables, such as cauliflower, broccoli, and most other vegetables.

Some foods may worsen symptoms of PASC, including processed foods with added sugar, foods high in simple carbohydrates such as breads and pastries; alcohol of any kind, and fried foods. Any foods that raise histamine levels may worsen symptoms of PASC, including: vinegar; tomato ketchup; tomatoes; eggplant; spinach; frozen/salted/canned fish (i.e., sardines and tune); fermented dairy products including cheese yogurt, sour cream, buttermilk, and kefir; fermented vegetables, such as sauerkraut and kimchi; pickles or pickled vegetables; kombucha; cured or fermented meats (i.e., sausages, salami, and fermented ham; wine, beer, champagne, and other alcohol products; fermented soy products, i.e. tempeh, miso, soy sauce; and fermented grains, such as sourdough bread.

In some embodiments, a therapeutic protocol for treating PASC includes consumption of sleep aids, such as California Poppy & Valerian root extract blend; cannabidiol (“CBD”), cannabinol (“CBN”), and Cannabis edibles; melatonin; and Benadryl.

Additional adjuncts to therapeutic protocols for treating PASC may include daily meditation, such as for between about twenty (20) and about forty (40) minutes; daily light exercise; stress avoidance; increased consumption of fluids, including at least about sixty-four (64) ounces of water daily and between two and four coups of Cistus incanus tea; Epsom salts baths three or four times weekly; acupuncture; and earthing/grounding.

PASC is a complex disease that affects multiple areas of the body and affects them in very different ways, therefore the therapeutic protocols disclosed herein are necessarily detailed and require ingestion of multiple natural substances. To address all of these complexities, the protocols require a broad spectrum of therapies, so following it effectively requires a certain fastidiousness. There are a lot of different ingredients and careful attention to dosing and timing is required.

In some embodiments, the therapeutic protocols disclosed herein may be condenses into a simple course of compressed tablets comprising the aforementioned active agents, which are taken orally

Embodiments of therapeutic protocols to treat PASC have been described herein. The PASC treatment protocols focus on a treatment to moderate a hyperimmune response; a treatment to destroy and remove the SARS CoV-2 spike protein from the gut and body, a treatment to detoxify the body and the brain; a treatment to replenish key nutrients; a treatment to mitigate depression and anxiety; and a regimen of physical and mental exercises. A shorter, three-week protocol is also disclosed for those with a milder form of PASC.

The embodiments and examples set forth herein were presented in order to best explain the present invention and its practical application, and to thereby enable those of ordinary skill in the art to make and use the invention. However, those of ordinary skill in the art will recognize that the foregoing description and examples have been presented for the purpose of illustration and example. The description as set forth is not intended to be exhaustive or to limit the invention to the precise form disclosed. Many modifications and variations are possible, in light of the teachings herein above.

### APPENDIX 1

### The Science and Purpose of Each Supplement

Andrographis paniculata

Andrographis is used in this protocol to help eliminate the over-reactive immune response and overall PASC symptoms in addition to helping eliminate the Covid S1 Spike protein. Andrographolide, the key medicinal component of Andrographis is well known for its broad-spectrum antiviral properties. Twenty-four studies showed andrographolide is effective against influenza A, hepatitis C virus, Chikungunya virus, HIV, hepatitis B virus, Herpes simplex virus 1, Epstein-Barr virus, and human papillomavirus. This study showed that both Andrographis paniculata extract and its active component andrographolide had a potent inhibitory effect against Covid-19.

https://www.ncbi.nlm.nih.gov/pmc/articles/PMC8056600/

Ashwagandha (Withania somnifera)

Ashwagandha is used in this protocol to help lower stress, anxiety, and depression, and improve sleep and overall brain health. It is also an adaptagenic used to raise the body's core energies.

It has a Cognition Promoting Effect and was useful in children with memory deficit and in old age people loss of memory. It was also found useful in neurodegenerative diseases such as Parkinson's, Huntington's and Alzheimer's diseases. It has xiolytic (anti-anxiety) effect and improves energy levels and mitochondrial health. It is an anti-inflammatory and anti-arthritic agent and was found useful in clinical cases of Rheumatoid and Osteoarthritis. https://www.ncbi.nlm.nih.gov/pmc/articles/PMC3252722/https://www.ncbi.nlm.nih.gov/pmc/articles/PMC6979308/

Black Seed Oil—Nigella sativa Drops

Nigella Sativa is used in this protocol to reduce the over-reactive immune response and help eliminate the PASC symptoms.

This has been extremely beneficial for me. The key ingredient is Thymoquinone which helps the body manage Mast Cell Activation Syndrome—also often part of PASC. The seed of Nigella sativa (N. sativa) has been used in different civilization around the world for centuries to treat various animal and human ailments. So far, numerous studies demonstrated the seed of Nigella sativa and its main active constituent, thymoquinone, to be medicinally very effective against various illnesses, including several chronic illnesses: neurological and mental illness, cardiovascular disorders, cancer, diabetes, inflammatory conditions, and infertility, as well as various infectious diseases due to bacterial, fungal, parasitic, and viral infections. https://www.ncbi.nlm.nih.gov/pmc/articles/PMC6535880/

### Black Walnut Hull

Black Walnut Hull is used in this protocol to help eliminate all remaining traces of the Covid-19 S1 spoke protein virus traces.

The Black Walnut hull has many key medicinal properties including triterpenoids, naphthoquinones, and α-tetralones, which have important antimicrobial, antiviral, anti-cancer, and antioxidant capabilities.

The walnut fruit includes four main parts: the kernel, skin, shell, and green husk. It has been demonstrated that the walnut green husk could be valued as a source of different natural bioactive compounds with excellent antioxidant and antimicrobial properties. https://www.ncbi.nlm.nih.gov/pmc/articles/PMC6719079/

Chaga Mushrooms (Inonotus obliquus)

Chaga is used in this protocol to help the Gut microbiome eradicate any spike protein remnants. This is done in conjunction with Turkey Tail mushrooms and prebiotics & probiotics.

Inonotus obliquus mushroom helps eliminate undesirable bacteria in the gut while increasing desirable bacteria. This helps prevent the spike protein from being able to hide in the gut microbiome helping to completely eliminate it. https://www.ncbi.nlm.nih.gov/pmc/articles/PMC5309192/

Cistus incanus Tea (Rockrose)

Cistus incanus is used in this protocol to help eliminate the over-reactive immune response and PASC symptoms, and help heal the upper respiratory tract, and lower whole-body inflammation. It also potentially plays apart in eliminating the spike protein from the body.

This tea has been extremely beneficial for me. And according to research, it's also a powerful antiviral drug, which also helps rid the body of HIV virus, Influenza A virus, Ebola virus and Marburg virus among others. “Cistus incanus extract combines broad antiviral activity with low risk of virus resistance. Its mechanism-of-action involves blocking of primary virus attachment to cells by selective targeting of the viral envelope glycoproteins. In contrast, many other natural products with broad-spectrum antiviral activity target host cell components. Our results demonstrate that multiple antiviral components within the Cistus incanus extract block virus attachment to the host cell. Altogether, the broad and potent in vitro antiviral effects of Cistus incanus extract demonstrated in this study support the further exploration of Cistus incanus extract and its components for novel antiviral approaches.” https://www.ncbi.nlm.nih.gov/pmc/articles/PMC4735868/

### Curcumin (Turmeric)

Curcumin is used in this protocol to help eliminate the over-reactive immune response and especially inflammation. Research suggests that curcumin can help in the management of oxidative and inflammatory conditions, metabolic syndrome, arthritis, anxiety, and hyperlipidemia (too much fat or cholesterol). It may also help in the management of exercise-induced inflammation and muscle soreness, thus enhancing recovery and subsequent performance in active people.

https://www.ncbi.nlm.nih.gov/pmc/articles/PMC5664031/

Desmodium molliculum (Burbur)

Burbur is used in this protocol to detox the kidneys, lymphatic system and protect the liver.

It is made from an extract from the leaves of Desmodium molliculum, a genus of the flowering plant family Fabaceae, a perennial that has been found and harvested in areas of Peru as high as 3,500 meters above sea level. Burbur leaf extract has been traditionally used for its body cleansing properties including the detoxification of the liver, kidneys, lymphatic system, and connective tissue.

https://pubmed.ncbi.nlm.nih.gov/23291573/

### EDTA (Calcium Disodium Ethylenediaminetetraacetic Acid)

EDTA is a chelation agent used in this protocol to help perform a final detox of the brain and the cleanup of heavy metals and other toxins leftover from the initial Covid infection, PASC and the healing process.

Neurotoxicity can be caused by numerous direct agents, of which toxic metals, organophosphorus pesticides, air pollution, radiation and electromagnetic fields, neurotoxins, chemotherapeutic and anesthetic drugs, and pathogens are the most important. Other indirect causes of neurotoxicity are cytokine and/or reactive oxygen species production and adoptive immunotherapy.

The chelating agent calcium disodium ethylenediaminetetraacetic acid (EDTA)—previously used to treat cardiovascular diseases—is known to be useful for the treatment of neurodegenerative diseases by removing these toxins.

https://pubmed.ncbi.nlm.nih.gov/30813622/

Ginseng (Panax quinquefolius)

Ginseng is used in this protocol to help rebuild the body's depleted core energy reserves. It also specifically helps the body heal from PASC via a variety of therapeutic effects, including antioxidation, anti-inflammatory, vasorelaxation, antiallergic, antidiabetic, and anticancer. Research results show that ginseng has beneficial effects on cardiac and vascular diseases through efficacy, including antioxidation, control of vasomotor function, modulation of ion channels and signal transduction, improvement of lipid profiles, adjustment of blood pressure, improvement in cardiac function, and reduction in platelet adhesion. https://www.ncbi.nlm.nih.gov/pmc/articles/PMC6026386/

### Gum Benzoin (Styrax)

Gum Benzoin is used in this protocol to help eliminate the leftover traces of the Covid-19 virus specifically the S1 spike protein.

It is used for many ailments and is known for its antibacterial, anti-biofilm and anticancer properties. The main medicinal ingredients are:
Benzioc Acid, which shows strong antiviral properties for Covid-19 https://www.ncbi.nlm.nih.gov/pmc/articles/PMC7770597/
Cinnamic Acid, which has shown to block Covid-19 via the active site of ACE2 receptor. https://pubmed.ncbi.nlm.nih.gov/33981493/
Pinoresinol, is extensively used as anticancer and antiviral agent. https://www.ncbi.nlm.nih.gov/pmc/articles/PMC8018901/

https://www.researchgate.net/publication/336825434_A_review_on_bioactive_potential_of_Benzoin_Resin

### Lion's Mane Mushroom

Hericium erinaceus (Lion's Mane) is used in this protocol to help protect and heal the brain and nervous system from any damage caused by the initial Covid-19 infection and PASC.

The evidence-based studies have shown that Lion's Mane can delay neuronal cell death with neurodegenerative diseases, such as ischemic stroke, Parkinson's disease, Alzheimer's disease and depression. Moreover, results have indicated that it can promote functional recovery and enhance nerve regeneration with neuropathic pain or presbycusis. https://www.ncbi.nlm.nih.gov/pmc/articles/PMC5987239/

### Liquid I.V.® (by Myogenix®)

Liquid IV is used in this protocol to keep the body fully hydrated and to have adequate electrolyte levels, so that the body and brain can heal more efficiently.

Electrolytes are essential for basic life functioning, such as maintaining electrical neutrality in cells, generating, and conducting action potentials in the nerves and muscles. Sodium, potassium, and chloride are the significant electrolytes along with magnesium, calcium, phosphate, and bicarbonates.

Liquid IV also uses 11 grams of non-GMO sugar to help force hydrate the body. At first, I was alarmed that I was also drinking sugar until I understood the science behind how it works. The sugar is part of Liquid IV's design in that it helps your body absorb water and electrolytes as quickly and efficiently as possible.

https://www.ncbi.nlm.nih.gov/books/NBK541123/

### N-Acetyl-L-Cysteine (NAC)

NAC is used in this protocol to help eliminate the over-reactive immune response and symptoms of PASC.

“We can take a lot of “antioxidants” but N-Acetyl-L-cysteine is the precursor to Glutathione which is the only antioxidant made in the body, and the only antioxidant that we know works intracellularly” according to Dr. William Lenaburg, MD.

NAC has antioxidant, anti-inflammatory and immune-modulating characteristics that may prove beneficial in the treatment and prevention of SARS-Cov-2. NAC can also boost the immune system, suppress viral replication, and reduce inflammation. Potential therapeutic benefits of NAC include, extracellularly scavenging ROS radicals, replenishing intracellular GSH, suppression of cytokine storm, and T cell protection, thus mitigating inflammation and tissue injury.

https://www.ncbi.nlm.nih.gov/pmc/articles/PMC7649937/

### Nicotinamide Riboside (NAD+)

Nicotinamide Riboside is used in this protocol to replenish NAD+ (nicotinamide adenine dinucleotide) levels which can be depleted by Covid-19. NMN is a viable alternative to nicotinamide riboside.

Low NAD+ levels can cause heart and neurological problems as well as slowing down cell regeneration, which is a key to healing. Low NAD+ levels can also lower serotonin levels which contribute to depression.

https://www.nature.com/articles/s42255-021-00507-3

There's also evidence suggesting that NAD may help the body repair neuronal stress or stress that occurs in the brain's neurons in animal test subjects. This indicates that NAD+ supplements may protect the brain against memory loss. Scientific research also suggests that NAD+ may increase endurance and strengthen the muscles, and lengthen lifespans by improving mitochondrial functions. https://www.nih.gov/news-events/news-releases/compound-repairs-neurological-damage-shows-cognitive-benefits-mouse-model-alzheimers-disease

Pimpinella anisum (Pinella)

Pinella, or Anise Oil, is used in this protocol to help detox the brain and nervous system, and lower inflammation.

An herbal extract made from Pimpinella anisum, commonly known as Anise, has been used by indigenous groups for centuries. Primary benefits of this cleanse include reducing inflammation, the elimination of toxins from both the brain and central nervous systems and reducing brain fog. Pimpinella anisum significantly reduces the quantity and severity of epileptic seizure attacks and helps protect the brain from trauma and lack of oxygen. https://www.ncbi.nlm.nih.gov/pmc/articles/PMC3416669/

### Psilocybin Mushroom

Psilocybin is used in this protocol to help heal the brain via its neurogenesis capabilities, reduce depression and anxiety via neuroplasticity, reduce brain fog, and help recover lost memories. It also can boost the immune system.

There is strong evidence showing substantial decreases in depression and anxiety symptoms that appear to persist for at least six months after a single active treatment of Psilocybin. Such results are unprecedented in psychiatry.

For example, according to studies found in the NIH library, no current pharmaceutical antidepressant works as effectively as Psilocybin over the long run. Psilocybin has also shown promising results for: Obsessive-Compulsive Disorder, Cluster Headaches, Addiction, Treatment-Resistant Depression, and Cancer-Related Psychiatric Distress. https://www.ncbi.nlm.nih.gov/pmc/articles/PMC5509636/

### Quercetin

Quercetin is used in this protocol to help eliminate the over-reactive immune response and overall PASC symptoms, especially inflammation, in addition to helping eliminate the Covid S1 Spike protein. Quercetin suppresses the NLRP3 inflammasome by affecting these regulators. Quercetin, as an anti-inflammatory, antioxidant, analgesic and inflammatory compound, is probably a potential treatment for severe inflammation and one of the main life-threatening conditions in patients with COVID-19. https://www.ncbi.nlm.nih.gov/pmc/articles/PMC7840793/

Quercetin also acts as a zinc ionophore. This means that quercetin transfers or pushes zinc into your body's cells, which prevents the cells from becoming infected. Zinc is an important mineral that can lessen the duration of colds and viruses.

Stevia rebaudiana

Stevia is used in this protocol to help breakdown Biofilms where the Covid-19 S1 spike protein can hide, and to help sweeten some of the medicines, especially the Cistus incanus tea.

Viruses and bacteria in biofilms are enclosed by an extracellular matrix that gives protection and improves survival of that virus or bacteria. Previous studies have shown that viruses such as Covid-19 can secondarily colonize preexisting biofilms, and viral biofilms have also been described.

Stevia extracts have effectively suppressed the activity of human retroviruses (HRV) by blocking the binding of virus with susceptible cells.

Unlike pharmaceutical antivirals like Remantadine, solutions of purified Stevia extract had marked virustatic and virucidal properties. https://www.ncbi.nlm.nih.gov/pmc/articles/PMC7089513/pdf/11094_2009_Article_270.pdf

Turkey Tail Mushrooms (Trametes versicolor)

Turkey Tail is used in this protocol to help the Gut microbiome eradicate any spike protein remnants. This is done in conjunction with Chaga mushrooms and prebiotics and probiotics. Trametes versicolor has been well recognized for its activity in maintaining the general health of the population and in managing and treating human diseases in various cultures. Polysaccharopeptide (PSP) is the active medicinal ingredient and has shown antitumorigenic and immunomodulatory effects, for example, elimination of prostate cancer stem cells and control of the intestinal microbiome, and its interplay with host cells as a prebiotic. https://pubmed.ncbi.nlm.nih.gov/27910783/

### Zeolite Clinoptilolite

Zeolite is used in this protocol to help detox the body and brain, and mop-up residual heavy metals, virus remnants and other toxins or poisons. It can also help keep the gut microbiome healthy.

Zeolite is a safe and excellent detoxifying, antioxidant, and anti-inflammatory agent. Zeolite, sometimes called “The Magic Stone,” is a porous volcanic mineral with high absorbency and ion-exchange capacity. The molecular structure is a dense network of Sodium Aluminate and Silicon-Oxygen Tetrahedrons that generates cavities where water and other polar molecules or ions are inserted/exchanged. https://pubmed.ncbi.nlm.nih.gov/26268084/https://protomfitness.co.uk/blog/2015/12/17/amazing-health-benefits-of-zeolites/

And here's interesting information from a company that sells Zeolite: https://www.therechargeclinic.com/post/everything-you-need-to-know-about-zeolite-for-detox

### Zinc

Zinc is used in this protocol to help reduce the over-reactive immune response and symptoms of PASC and is used as an antiviral therapy and immunomodulatory agent.

It directly inhibits viral replication and balances the immune response during infectious diseases. Quercetin and Zinc work very well together to prevent and stop infection. Quercetin somehow makes it so that Zinc can enter cells more easily, thereby protecting many more cells from getting infected, slowing down the infection.

Zinc supplementation also improves the mucociliary clearance (lung defense), strengthens the integrity of the epithelium (layers of protective cells), decreases viral replication, preserves antiviral immunity, attenuates the risk of hyper-inflammation, supports anti-oxidative effects and thus reduces lung damage and minimized secondary infections. https://www.ncbi.nlm.nih.gov/pmc/articles/PMC7365891/.

### APPENDIX II

### Myovite® Nutrient List

### Vitamins & Minerals:

Vitamin A (as Beta Carotene) 10,000 iu
Vitamin C (as Ascorbic Acid) 1,000 mg
Vitamin D (as Cholocalciforol) 5,000 iu
Vitamin E (as D-Alpha Tocopherol Acetate) 400 iu
Vitamin B1 (as Thiamin HCl) 50 mg
Vitamin B2 (as Riboflavin) 50 mg
Niacin (as Niacinamido) 20 mg
Vitamin B6 (as Pyridoxine HCl) 50 mg g
Folate (as Folic Acid) 800 mcg
Vitamin B12 (as Methylcobalamin) 250 mcg
Biotin 600 mcg
Vitamin B5 (as Pantothenic Acid) 50 mg
Calcium (as Calci-K Albion) 90 mg
Phosphorus (as Calci-K Albion 45 mg
Iodine (from Kelp Ascophylum nododom) 100 mcg mg
Magnesium (as Magnesium Glycinate Cholato and Magnesium Glycyl Glutamine Albion) 234 mg
Zinc (as Zinc Cholazome Amino Acid Cholato Albion) 30 mg
Selenium (as Selenium Amino Acid Chelatexalbion) 200 mcg
Copper (as Copper Cholazome Amino Acid Cholate]Albion) 2 mg
Manganese (as Manganese Chelazome Amino Acid Chelate Albion) 4 mg
Chromium (as Chromium Chelavite Amino Acid Chelate](Albion) 300 mcg
Potassium (as Calci-K] Albion) 80 mg

### ProEnzyme Plus Probiotics Proprietary Blend 327 mg

Pro Enzyme Digestive Enzyme Blend:
Proteasco 49,500 HUT's
Amylase 3,300 SKB's
Lipase 1,170 FIP's
Cellulase 4,725 CU's
Lactospore Lactobacillus Sporogenes (Bacillus Coagulans/Lactic Acid Bacteria 100 to 200 million organisms per serving)
Shiitake Mushroom Extract (whole)
Reishi Mushroom 10:1 Extract (whole)
Maritake Mushroom (whole)
Aribinogalactin (larch tree)

### Synerchi Green Food Nutrients Proprietary Blend 1521 mg

Pure Barley Grass Juice Powder (horduem vulgare)(young leaves) (100% Organic and Kosher, Cold Temperature Dried,
Chlorophyll 9.1 mg/g
Beta-Galactosidase enzyme 1,000 units/g
Beta-Glucosidase enzyme 300 units/g
Acid-Phosphatase enzyme 2,300 units/g
Alpha-Mannosidase enzyme 2,100 units/g
Superoxide-Dismutase enzyme 6,200 units/g]
Pure Chlorella (Chlorella regularis) (Hermetically Sealed Tank Grown, Soft Cell Wall Pharmaceutical and Kosher
Chlorophyll 1.7 mg/g)

### CardioVascular Support Proprietary Blend 241 mg

Alpha Lipoic Acid (as Thioctic Acid)
Coenzyme Q10 (CO Q 10) (as Ubidecaronone)
Grape Seed Extract (95% Proanthocyanidins](Vitis vinifera)]seed)
Acai Berry 4:1 Extract (Euterpe oleracae](fruit)
Resveratrol 50% (from Japanese Knotwood) (Polygonum cuspidatum](root)
Garlic (Alium sativum)[bulb)

### Performance Optimizers Proprietary Blend 2302 mg

Beta Alanine
Magnesium Glycyl Glutamine Cholate (Albion)
Siberian Rhodiola Rosea (5% Rosavirus, 3% Salidrosides)(root)
Ashwagandha Root Extract (Withania somnifera)[5% Withanolides)
Natural Brazilian Guarana (4% Guaranine)[Paulinia cupana)(seed)

### Liver Detox Proprietary Blend 511 mg

N-Acetyl L-Cysteine
Guggul Herb Extract (10% Guggulsterones](Commiphora mukul](gum)
Phosphatidyl Choline 20%
Milk Thistle Seed Extract (80% Sllymarin)[Slybum marianum)
Dandelion Root
L-Methionine
Tumeric Root Extract (95% Curcuminoids)(Curcoma longa)
Artichoke Leaf 4:1 Extract (Cynara scolymus)
Burdock Root 4:1 Extract (Arctium lappa)
Hydrangea Root 4:1 Extract (Hydrangea arboresescens)
Turkey Tail Mushroom 4:1 Extract (Trametes versicolor](whole)
Yarrow Flower (Achillea millefolium)

### Joint & Tissue Support Proprietary Blend 1503 mg

Opti-MSM Methyl Sulfonyl Methane (MSM)
Glucosamine Sulfate KCl
Cissus Quandrangularis 4:1 Extract (Cissus quandrangularis)(whole)
Chondroitin Sulfate
Bromelain (2400 gdu/g)

What is claimed is:

1. A method for treating post-Covid syndrome comprising:
administering a first compositional blend comprising (i) an infusion (tea) of Cistus incanus, (ii) quercetin, (iii) Andrographis paniculata, (iv) nicotinamide mononucleotide, and (v) curcumin;
administering a second composition comprising gum benzoin, Jugulans nigra hull, Inonotus obliquus, and Trametes versicolor;
administering a third composition comprising zeolite, Desmodium molliculum, and Pimpinella anisum;
administering a fourth composition comprising nicotinamide riboside, pterostilbene, Panax quinquefolius, and Withania somnifera; and
administering a fifth composition comprising psilocybin and Hericium erinaceus.

2. The method of claim 1, wherein the first composition, the second composition, the third composition, and fourth composition, and the fifth composition are administered daily for three (3) consecutive weeks.

3. The method of claim 1, wherein the first composition, the second composition, the third composition, and fourth composition, and the fifth composition are administered daily for six (6) consecutive weeks.

4. The method of claim 1, wherein the first composition additionally comprises zinc.

5. The method of claim 1, wherein the first composition additionally comprises Nigella sativa.

6. The method of claim 1, wherein the quantity of quercetin is 1,600 milligrams.

7. A method of claim 1, wherein the second composition additionally comprises ethylenediaminetetraaceitic acid (EDTA).

8. The method of claim 1, wherein the third composition additionally comprises Zeolite.

9. The method of claim 1, wherein the third composition additionally comprises Slybum marianum.

10. The method of claim 1, wherein the fourth composition additionally comprises a multivitamin blend having at least three of the vitamins and minerals selected from the group of vitamins and minerals consisting essentially of Vitamin C, Vitamin D, Vitamin B complex, magnesium, selenium, and mixtures thereof.

11. The method of claim 8, wherein the fourth composition additionally comprises chlorophyll.

12. The method of claim 1, wherein the quantity of nicotinamide riboside is 300 milligrams.

13. The method of claim 1, wherein the quantity of pterostilbene is 100 milligrams.

14. The method of claim 1, additionally comprising administration of a sixth composition comprising Astragalus membranaceous and Cordyceps sinensis.

15. The method of claim 2, wherein the quantity of quercetin is 2,000 milligrams.

16. The method of claim 2, wherein the quantity of Andrographis paniculata is 2,000 milligrams.

17. The method of claim 2, wherein the quantity of Hericium erinaceus is 2,000 milligrams.

18. The method of claim 2, wherein the quantity of Withania somnifera is 2,600 milligrams.

19. A method for treating post-Covid syndrome comprising administration of a fungus genus selected from the group of fungi genera consisting essentially of Reishi, Shiitake, and Maritake and mixtures thereof.

20. The method of claim 19, wherein the fungus genus is administered orally one time each day.
